(19) United States
(12) Patent Application Publication
(10) Pub. No.: US 2010/0210871 A1
(43) Pub. Date: Aug. 19, 2010

(54) Reactive Extraction of Free Organic Acids from the Ammonium Salts Thereof

(75) Inventors: Christoph Kobler, Alzenau (DE); Dieter Buss, Aschaffenburg (DE); Axel Ronneburg, Erlensee (DE); Christoph Weckbecker, Grundau-Lieblos (DE)

Correspondence Address: LAW OFFICE OF MICHAEL A. SANZO, LLC, 15400 CALHOUN DR., SUITE 125, ROCKVILLE, MD, 20855, US

(73) Assignee: EVONIK DEGUSSA GMBH, Essen (DE)

(21) Appl. No.: 12/706,512

(22) Filed: Feb. 16, 2010

Related U.S. Application Data
(60) Provisional application No. 61/239,634, filed on Sep. 3, 2009.

(30) Foreign Application Priority Data
Feb. 19, 2009 (DE) 102009001008.4

(51) Int. Cl. C07F 9/535; C07C 61/00; C07C 59/01; C07C 59/10; C07C 53/126; C07C 309/25

(52) U.S. Cl. 562/8; 562/400; 562/579; 562/587; 562/512; 562/100

(57) ABSTRACT

The invention relates to a process for converting ammonium salts of organic acids to the particular free organic acid, wherein an aqueous solution of the ammonium salt is contacted with an organic extractant and the salt is dissociated at temperatures and pressures at which the aqueous solution and the extractant are in the liquid state, and a stripping medium or entraining gas is introduced in order to remove NH₃ from the aqueous solution and transfer at least a portion of the free organic acid formed to the organic extractant. The invention described here thus provides an improved process for releasing an organic acid, preferably a carboxylic, sulphonic or phosphonic acid, especially an alpha-hydroxycarboxylic acid or beta-hydroxycarboxylic acid, from the ammonium salt thereof by release and removal of ammonia and simultaneous extraction of the acid released with a suitable extractant from the aqueous phase. This process corresponds to a reactive extraction. The reactive extraction of an organic acid from the aqueous ammonium salt solution thereof can be improved significantly by the use of a stripping medium or entraining gas, for example nitrogen, air, steam or inert gases, for example argon. The ammonia released is removed from the aqueous solution by the continuous gas stream and can be fed back into a production process. The free acid can be obtained from the extractant by a process such as distillation, rectification, crystallization, re-extraction, chromatography, adsorption, or by a membrane process.

### CROSS REFERENCE TO RELATED APPLICATIONS

The present application claims the benefit of U.S. provisional application 61/239,634 filed on Sep. 3, 2009, and priority to German Application (DE) 102009001008.4, filed on Feb. 19, 2009.

### FIELD OF THE INVENTION

The present invention relates to a novel, improved process for preparing and isolating free organic acids such as carboxylic acids, sulphonic acids, phosphonic acids and especially alpha-hydroxycarboxylic acids from the corresponding ammonium salts thereof

### BACKGROUND OF THE INVENTION

Organic acids include the group of the substituted carboxylic acids (I-III), sulphonic acids (IV) and phosphonic acids (V):
monocarboxylic acid:

X¹—COOH I
dicarboxylic acid:

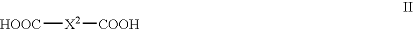

tricarboxylic acid:

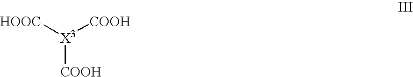

sulphonic acid:

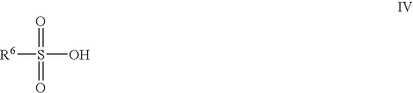

phosphonic acid:

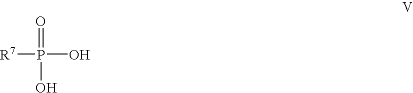

Hydroxycarboxylic acids are specific carboxylic acids which possess both a carboxyl group and a hydroxyl group. Most naturally occurring representatives are alpha-hydroxycarboxylic acids, i.e. the hydroxyl group is on a carbon atom adjacent to the carboxyl group.

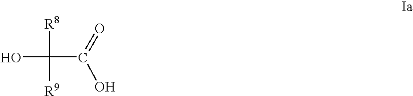

Important alpha-hydroxycarboxylic acids are, as well as lactic acid, glycolic acid, citric acid and tartaric acid, also 2-hydroxyisobutyric acid as a precursor of methacrylic acid and methacrylic esters. These find their main field of use in the preparation of polymers and copolymers with other polymerizable compounds. An alpha-hydroxycarboxylic acid which is likewise commercially important is 2-hydroxy-4-methylthiobutyric acid, which is typically referred to as methionine hydroxy analogue (MHA), and plays an important role in animal nutrition in addition to the essential amino acid methionine, in particular in the case of monogastric animals, for example poultry and pigs. Racemic MHA can be used directly as an animal feed additive, since there is a conversion mechanism under in vivo conditions in some species, which converts both enantiomers of MHA to the natural amino acid L-methionine. This involves first oxidizing the 2-hydroxy-4-methylthiobutyric acid with the aid of an unspecific oxidase to a-ketomethionine, and then converting it further with an L-transaminase to L-methionine. This increases the available amount of L-methionine in the organism, which can then be available to the animal for growth.

A further class of hydroxycarboxylic acids is that of the beta-hydroxycarboxylic acids with the general formula Ib:

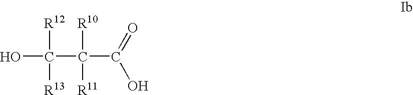

Important beta-hydroxycarboxylic acids are, for example, 3-hydroxypropionic acid, 3-hydroxybutyric acid, 3-hydroxyvaleric acid, 3-hydroxyhexanoic acid and 3-hydroxy-isobutyric acid. Just like 2-hydroxyisobutyric acid, the latter can likewise serve as a precursor for the industrially important products methacrylic acid and methacrylic esters. All organic acids form the corresponding ammonium salts with ammonia, for example according to the general formula of the monocarboxylic acid:

X¹—COO⁻NH₄⁺

According to the state of the art, alpha-hydroxycarboxylic acids are preferably prepared from their parent cyanohydrins with the aid of mineral acids, for example hydrochloric acid, phosphoric acid or preferably with sulphuric acid. To isolate the free acid, only the mineral acid used for hydrolysis is neutralized subsequently with a base, preferably ammonia. All of the mineral acid and the base used for neutralization are necessarily obtained in these processes in at least stoichiometric and hence very large amounts in the form of mineral salts, usually in the form of ammonium sulphate. These salts can be disposed of on the market only with difficulty and only with losses compared to the feedstocks. Owing to this problem, large amounts of these salts even have to be disposed of with disposal costs.

Another chemical process is the hydrolysis of cyanohydrin with inorganic bases, for example sodium hydroxide. It is likewise necessary here to release the alpha-hydroxycarboxylic acid by adding a mineral acid in stoichiometric amounts. The hydrolysis of cyanohydrins likewise goes to the stage of the ammonium salt with titanium dioxide as the catalyst. The salt problem remains the same.

Mono-, di- and tricarboxylic acids, and alpha- and beta-hydroxycarboxylic acids, can be prepared fermentatively with the aid of microorganisms or enzymatically. This affords the organic acids in the form of the ammonium salt. They are released by adding the stoichiometric amount of a mineral acid. In the case of di- or tricarboxylic acids, even twice or three times the stoichiometric amount of a mineral acid has to be added. This likewise gives rise to very large amounts of ammonium salts, which in turn have to be recycled in a complex manner or disposed of expensively.

Processes in which no salt burden occurs are to date uneconomic on the industrial scale for reasons of cost. One example thereof is the esterification of an ammonium salt of an alpha-hydroxycarboxylic acid with an alcohol and subsequent hydrolysis of the ester with an acid catalyst (JP7194387).

In order to prepare free carboxylic acids from the ammonium salts, there are various processes based on the thermal decomposition of the ammonium carboxylates to release ammonia (Scheme 1):

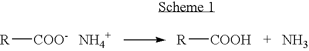

According to GB967352, a small amount of water is added to an ammonium salt of an unsaturated fatty acid, and the mixture is heated at total reflux (80° C.) or higher in organic solvents in order to free or to remove ammonia to obtain the unsaturated fatty acid.

According to JP54115317, an organic solvent which forms an azeotropic mixture with water is added to a 10-50% aqueous solution of ammonium methacrylate, and the solution which arises is heated to 60-100° C. As a result, water is distilled off as an azeotropic mixture and ammonia is removed simultaneously in order to obtain free methacrylic acid.

According to JP7330696, a 10-80% aqueous solution of an ammonium salt of an acidic amino acid is heated with addition of water. Ammonia and water are distilled off and the amino acid is released.

In these processes, ammonia is in principle removed easily, since the carboxylic acid has a high dissociation constant. In contrast, the degree of dissociation of ammonium ions from ammonium salts of carboxylic acids with pKₐ values less than 4, for example sulphonic acids and alpha-hydroxycarboxylic acids, is low. It is therefore very difficult to remove ammonia from the salts of strong acids. In order to remove the majority of ammonia, a long period is required, or it is necessary to add a large amount of water or of organic solvents. In the abovementioned processes, 50% or more of the corresponding carboxylic acid remains as the ammonium salt.

U.S. Pat. No. 6,066,763 describes a process for preparing alpha-hydroxycarboxylic acids, which proceeds without the inevitable occurrence of large amounts of salts which can be disposed of only with difficulty, if at all. In this process, the starting materials used are the ammonium salts of the corresponding alpha-hydroxycarboxylic acids, said salts being obtainable from the corresponding cyanohydrins with the aid of enzymes (nitrilases). The salt is heated in the presence of water and a solvent. Preferred solvents have a boiling point of >40° C. and form an azeotrope with water. The distillative removal of the azeotropic mixture releases ammonia, which escapes in gaseous form via the condenser. The corresponding alpha-hydroxycarboxylic acid accumulates in the bottom of the distillation apparatus. However, the removal of the water at elevated temperature results in large amounts of the initially released alpha-hydroxycarboxylic acid being converted by intra- or else intermolecular esterification to dimers and polymers of the alpha-hydroxycarboxylic acid in question. These subsequently have to be converted back to the monomeric alpha-hydroxycarboxylic acid in question by heating with water under elevated pressure. Another disadvantage is the long residence times in both process stages. They are 4 hours in the examples mentioned. Since the solvent is kept at boiling for the whole time in stage 1, the steam consumption is uneconomically high. The cause of this is the release of the alpha-hydroxycarboxylic acid, which becomes more difficult with increasing depletion of ammonia. It does not succeed 100%. After the end of the reaction, 3-4% bound ammonia still remains in the bottoms. Under the reaction conditions, another by-product which occurs is the corresponding amide of the alpha-hydroxycarboxylic acid, which is only partly converted to the corresponding ammonium salt by hydrolysis in stage 2 of the process (Scheme 2).

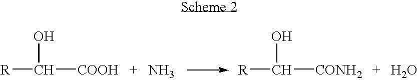

The alpha-hydroxycarboxylic acids obtained possess only a purity of approx. 80%, and so a further purification by means of liquid-liquid extraction or crystallization is usually necessary.

In patent publication WO 00/59847, the ammonium salt solutions of the alpha-hydroxycarboxylic acids are brought to a concentration of >60% under reduced pressure. The conversion to dimeric or polymeric esters of the corresponding alpha-hydroxycarboxylic acids is intended to be less than 20%. Passing a gas through, preferably steam, releases and drives out ammonia. Using the example of 2-hydroxy-4-methylthiobutyric acid, 70% free acid is achieved; the remainder consists of the unconverted ammonium salt of 2-hydroxy-4-methylthiobutyric acid and the corresponding dimeric ester.

US 2003/0029711 A1 describes a process for obtaining organic acids, including from aqueous solutions of the ammonium salts, with addition of a hydrocarbon as an entraining agent. Heating the mixture affords a gaseous product stream which comprises an azeotrope consisting of the organic acid and the entraining agent. In order to isolate the acid from this product stream, further steps such as condensation and additional distillations must be carried out. Furthermore, this process also requires the addition of additional chemicals (entraining agents), which makes the process significantly more costly, specifically for use on the industrial scale.

U.S. Pat. No. 6,291,708 B1 describes a process in which an aqueous solution of an ammonium salt is mixed with a suitable alcohol, and this alcohol-water mixture is then heated under elevated pressure in order to decompose the ammonium salt thermally to the free acid and ammonia. At the same time, a suitable gas as an entraining agent is contacted with the alcohol-water mixture, so as to drive out a gaseous product stream comprising ammonia, water and a portion of the alcohol, while at least 10% of the alcohol remains in the liquid phase and reacts with the free acid to give the corresponding ester. The disadvantages of this process include the necessity of additional chemicals (alcohol and a gas as an entraining agent) and the partial conversion of the free carboxylic acid formed to the ester, which in turn has to be hydrolysed in order to obtain the free carboxylic acid.

In DE 10 2006 052 311 A1 (published specification), the ammonium salt of an alpha-hydroxycarboxylic acid is heated in the presence of a tertiary amine with release of the ammonia and formation of the salt of tertiary amine and alpha-hydroxycarboxylic acid in question. Subsequently, the salt is dissociated thermally and the tertiary amine formed is recovered by distillation. The free alpha-hydroxycarboxylic acid remains in the distillation bottoms. The purity of the alpha-hydroxycarboxylic acids obtained is 95%.

In DE 10 2006 049 767 A1 (published specification), this process is applied to the preparation of 2-hydroxy-4-methylthiobutyric acid from the corresponding 2-hydroxy-4-methylthiobutyramide. With N-methylmorpholine, 2-hydroxy-4-methylthiobutyric acid is formed at 180° C. and 6 bar in a purity of 95% with 96% yield. The use of other tertiary amines gives similar results.

In DE 10 2006 049 768 A1 (published specification), the 2-hydroxy-4-methylthiobutyramide formed by mineral acid hydrolysis of 2-hydroxy-4-methylthiobutyronitrile is extracted with a polar water-immiscible solvent. Preferred solvents are ethers, ketones and trialkylphosphine oxides, also in mixtures with various hydrocarbons. The solvent is removed by distillation and the resulting 2-hydroxy-4-methylthiobutyramide is base-hydrolysed. The bases used are tertiary amines, which can be removed again by distillation from the salts formed with release of the 2-hydroxy-4-methylthiobutyric acid. The temperatures in this process are between 130 and 180° C. at 6 bar.

The disadvantages of the latter processes are the use of tertiary amines as additives. These cannot be removed completely by distillation and thus remain in a small amount in the end product. The high temperatures of 130 to 180° C. employed are not very economic and the pressure range of 6 bar requires increased capital costs in the industrial implementation.

In U.S. Pat. No. 6,815,560 and the patent publications cited there, the free 2-hydroxy-4-methylthiobutyric acid prepared by sulphuric acid hydrolysis is extracted from the hydrolysis solution with a water-immiscible solvent, preferably isobutyl methyl ketone. The extractant is recovered by distillation; the 2-hydroxy-4-methylthiobutyric acid remains in the distillation bottoms in its monomeric and dimeric forms. The addition of water adjusts the thermodynamic equilibrium between the two forms.

### SUMMARY OF THE INVENTION

Against the background of the disadvantages of the prior art, it was an object of the present invention to find an inexpensive and environmentally compatible process for isolating free organic acids such as carboxylic acids, sulphonic acids, phosphonic acids and especially alpha- and beta-hydroxycarboxylic acids from the ammonium salts thereof, which does not form a salt burden as a coproduct and is completely back-integrated by means of closed circuits.

The technical object is achieved by a process for converting ammonium salts of organic acids to the particular free organic acid, wherein an aqueous solution of the ammonium salt is contacted with an organic extractant and the salt is dissociated at temperatures and pressures at which the aqueous solution and the extractant are in the liquid state, and a stripping medium or entraining gas is introduced in order to remove NH₃ from the aqueous solution and transfer at least a portion of the free organic acid formed to the organic extractant.

The invention thus provides a process wherein the ammonium salt of organic acids is converted by means of reactive extraction using a stripping medium or entraining gas, for example by driving out (stripping) the ammonia with steam or nitrogen, to the free organic acid which is then transferred into the organic extractant. It is preferred that at least 50%, preferably at least 80%, more preferably at least 90% and most preferably at least 95% of the free organic acid formed is transferred into the organic extractant.

In a preferred process, the conversion is effected at pressures of 0.01 bar to 200 bar, particularly of 0.01 bar to 20 bar, very preferably of 0.1 bar to 5 bar. Moreover, it is preferred that the salt dissociation is performed at temperatures of 5° C. to 300° C., more preferably of 20° C. to 300° C., even more preferably of 40° C. to 200° C., especially preferably of 50° C. to 130° C.

The temperature has a high influence on the rate of formation of the free acid and the end yield thereof. The temperature is guided by the extractant used and is, according to the invention, below the boiling point of the aqueous solution or of a possible azeotrope, the boiling point of the aqueous solution or of any azeotrope which forms depending of course on the particular pressure applied.

As already described above, the salt dissociation in the process according to the invention is performed at temperatures and pressures at which the aqueous solution and the extractant are in liquid form, not in solid form and not in gaseous form, i.e. below the boiling temperature, which depends on the particular pressure applied, of the aqueous solution or of any azeotropic mixture which forms.

According to the invention, the starting concentration of the ammonium salt of the organic acid in the aqueous solution used is preferably in the range from 90% by weight to 1% by weight, more preferably from 75% by weight to 5% by weight and most preferably from 60% by weight to 10% by weight. In the course of the reaction of the salt dissociation, the corresponding concentration of the salt decreases.

Moreover, it is preferred that the extractant used is a sparingly water-miscible or entirely water-immiscible solvent. The weight ratio of aqueous solution and organic extractant is from 1:100 to 100:1, more preferably from 1:10 to 10:1, most preferably from 1:5 to 5:1. According to the present invention, the organic acid may be selected from the group of monocarboxylic acid, dicarboxylic acid, tricarboxylic acid, ascorbic acid, sulphonic acid, phosphonic acid, hydroxycarboxylic acid, especially alpha-hydroxycarboxlic acid or beta-hydroxycarboxylic acid.

In further process steps, according to the invention, after the salt dissociation has ended, the organic acid formed can be obtained from the organic extractant.

In a preferred embodiment, the organic acid corresponds to a carboxylic acid of the general formula I

X¹—COOH I

where X¹ is an organic radical selected from the group comprising unsubstituted and mono- or polysubstituted, branched and straight-chain alkyl radicals, cycloalkyl radicals, alkenyl radicals having one or more double bonds, alkynyl radicals having one or more triple bonds, aryl radicals, alkylaryl radicals, arylalkyl radicals, arylalkenyl radicals, alkyloxyalkyl radicals, hydroxyalkyl radicals and alkylthioalkyl radicals.

In one alternative, it is preferred that X¹ is an organic radical selected from the group of (C₁-C₁₈)-alkyl radicals, (C₃-C₁₈)-cycloalkyl radicals, (C₂-C₂₆)-alkenyl radicals having one or more double bonds, (C₂-C₂₆)-alkynyl radicals having one or more triple bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, (C₆-C₁₀)-aryl-(C₂-C₂₆)-alkenyl radicals, (C₁-C₁₈)-alkyloxy-C₁-C₁₈)-alkyl radicals, (C₁-C₁₈)-hydroxyalkyl radicals and (C₁-C₁₈)-alkylthio-(C₁-C₁₈)-alkyl radicals.

In another alternative, X¹ is preferably CR¹R²R³ where R¹=H, OH, OR⁴, NH₂, NHR⁴, NR⁴R⁵, Cl, Br, I, F, where R², R³, R⁴ and R⁵ are each independently selected from the group comprising H, unsubstituted and mono- or polysubstituted, branched and straight-chain (C₁-C₁₈)-alkyl radicals, (C₃-C₁₈)-cycloalkyl radicals, (C₂-C₂₆)-alkenyl radicals having one or more double bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, especially benzyl radicals, (C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals.

The organic acid is preferably selected from the group of acetic acid, propionic acid, butyric acid, valeric acid, caproic acid, oenanthic acid, caprylic acid, pelargonic acid, capric acid, lauric acid, palmitic acid, stearic acid, omega-3 fatty acids such as linolenic acid, omega-6 fatty acids such as linoleic acid and arachidonic acid, omega-9 fatty acids such as oleic acid and nervonic acid, salicylic acid, benzoic acid, ferulic acid, cinnamic acid, vanillic acid, gallic acid, hydroxycinnamic acids, hydroxybenzoic acids, 3-hydroxypropionic acid.

In an alternative process the organic acid corresponds to a dicarboxylic acid of the general formula II

HOOC—X²—COOH II

where X² is an organic radical selected from the group comprising unsubstituted and mono- or polysubstituted, branched and straight-chain alkanediyl radicals, cycloalkanediyl radicals, alkenediyl radicals having one or more double bonds, alkynediyl radicals having one or more triple bonds, aryldiyl radicals, alkylaryldiyl radicals, arylalkanediyl radicals, arylalkenediyl radicals, alkyloxyalkanediyl radicals, hydroxyalkanediyl radicals and alkylthioalkanediyl radicals.

The suffix “-diyl” indicates in this context that both carboxylic acid groups of the dicarboxylic acid are bonded to this radical. The carboxylic acid groups may each independently be bonded to any carbon atoms of the organic radical, for example geminally, vicinally or to nonadjacent carbon atoms, and the carbon atoms to which the carboxylic acid groups are bonded may either be in terminal positions or within the radical.

It is preferred that X² is defined as follows: an organic radical selected from the group of unsubstituted and mono- or polysubstituted, branched and straight-chain (C₁-C₁₈)-alkanediyl radicals, (C₃-C₁₈)-cycloalkanediyl radicals, (C₂-C₂₆)-alkenediyl radicals having one or more double bonds, (C₂-C₂₆)-alkynediyl radicals having one or more triple bonds, (C₆-C₁₀)-aryldiyl radicals, especially phenyldiyl radicals, (C₁-C₁₈)-alkyl-(C₆-C₁₀)-aryldiyl radicals, (C₆-C₁₀)-aryl-(C₁-C₁₈)-alkane diyl radicals, (C₆-C₁₀)-aryl-(C₂-C₂₆)-alkenediyl radicals, (C₁-C₁₈)-alkyloxy-(C₁-C₁₈)-alkanediyl radicals, (C₁-C₁₈)-hydroxyalkanediyl radicals and (C₁-C₁₈)-alkylthio-(C₁-C₁₈)-alkanediyl radicals, any substituents being selected from the group comprising OH, OR⁴, NH₂, NHR⁴, NR⁴R⁵, Cl, Br, I and F, where R⁴, R⁵ are each independently selected from the group comprising H, unsubstituted and mono- or polysubstituted, branched and straight-chain (C₁-C₁₈)-alkyl radicals, (C₃-C₁₈)-cycloalkyl radicals, (C₂-C₂₆)-alkenyl radicals having one or more double bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, especially benzyl radicals,(C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals.

The organic acid is preferably selected from the group of succinic acid, oxalic acid, malonic acid, glutaric acid, adipic acid, pimelic acid, suberic acid, azelaic acid, sebacic acid, fumaric acid, itaconic acid, methylmalonic acid, phthalic acid, terephthalic acid, isophthalic acid.

In a further alternative process the organic acid is a tricarboxylic acid of the general formula III

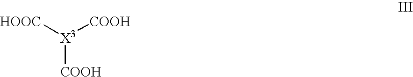

where X³ is an organic radical selected from the group comprising unsubstituted and mono- or polysubstituted, branched and straight-chain alkanetriyl radicals, cycloalkanetriyl radicals, alkenetriyl radicals having one or more double bonds, alkynetriyl radicals having one or more triple bonds, aryltriyl radicals, alkylaryltriyl radicals, arylalkanetriyl radicals, arylalkenetriyl radicals, alkyloxyalkanetriyl radicals, hydroxyalkanetriyl radicals and alkylthioalkanetriyl radicals.

The suffix “-triyl” indicates here that the three carboxylic acid groups of the tricarboxylic acid are bonded to this radical. The carboxylic acid groups may each where R⁶ is an organic radical selected from the group comprising unsubstituted and mono- or polysubstituted, branched and straight-chain alkyl radicals, cycloalkyl radicals, alkenyl radicals having one or more double bonds, alkynyl radicals having one or more triple bonds, aryl radicals, alkylaryl radicals, arylalkyl radicals, arylalkenyl radicals, alkyloxyalkyl radicals, hydroxyalkyl radicals and alkylthioalkyl radicals.

It is preferred that R⁶ is defined as follows: unsubstituted and mono- or polysubstituted, branched and straight-chain (C₁-C₁₈)-alkyl radicals, (C₃-C₁₈)-cycloalkyl radicals, (C₂-C₂₆)-alkenyl radicals having one or more double bonds, (C₂-C₂₆)-alkynyl radicals having one or more triple bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, (C₂-C₂₆)-alkenyl radicals, (C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals, any substituents being selected from the group comprising OH, OR⁴, NH₂, NHR⁴, NR⁴R⁵, Cl, Br, I and F, where R⁴ and R⁵ are each independently selected from the group comprising H, unsubstituted and mono- or polysubstituted, branched and straight-chain (C₁-C₁₈)-alkyl radicals, (C₃-C₁₈)-cycloalkyl radicals, (C₂-C₂₆)-alkenyl radicals having one or more double bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, especially benzyl radicals,(C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals.

In a preferred process, the organic acid is selected from the group of p-toluenesulphonic acid, camphor-10-sulphonic acid, benzenesulphonic acid, dodecylbenzenesulphonic acid, naphthalenesulphonic acids, phenolsulphonic acids.

In a further process according to the present invention, the organic acid is a phosphonic acid of the general formula V

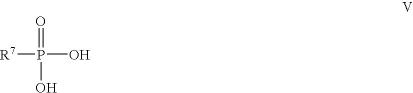

independently be bonded to any carbon atoms of the organic radical, for example geminally, vicinally or to nonadjacent carbon atoms, where the carbon atoms to which the carboxylic acid groups are bonded may either be in terminal positions or within the radical.

Moreover, it is preferred that X³ is defined as follows: unsubstituted and mono- or polysubstituted, branched and straight-chain (C₁-C₁₈)-alkanetriyl radicals, (C₃-C₁₈)-cycloalkanetriyl radicals, (C₂-C₂₆)-alkenetriyl radicals having one or more double bonds, (C₂-C₂₆)-alkynetriyl radicals having one or more triple bonds, (C₆-C₁₀)-aryltriyl radicals, especially phenyltriyl radicals, (C₁-C₁₈)-alkyl-(C₆-C₁₀)-aryltriyl radicals, (C₆-C₁₀)-aryl-(C₁-C₁₈)-alkanetriyl radicals, (C₆-C₁₀)-aryl-(C₂-C₂₆)-alkenetriyl radicals, (C₁-c₁₈-alkyloxy-(C₁-C₁₈)-alkanetriyl radicals, (C₁-C₁₈)-hydroxyalkanetriyl radicals and (C₁-C₁₈)-alkylthio-(C₁-C₁₈)-alkanetriyl radicals, any substituents being selected from the group comprising OH, OR⁴, NH₂, NHR⁵, NR⁴R⁵, Cl, Br, I and F, where R⁴, R⁵ are each independently selected from the group comprising H, unsubstituted and mono- or polysubstituted, branched and straight-chain (C₁-C₁₈)-alkyl radicals, (C₃-C₁₈)-cycloalkyl radicals, (C₂-C₂₆)-alkenyl radicals having one or more double bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, especially benzyl radicals,(C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals.

In a preferred embodiment, the organic acid is selected from the group of citric acid, cyclopentane-1,2,3-tricarboxylic acid, cyclopentane-1,2,4-tricarboxylic acid, 2-methylcyclopentane-1,2,3-ticarboxylic acid, 3-methylcyclopentane-1,2,4-tricarboxylic acid.

In a further process, the organic acid corresponds to a sulphonic acid of the general formula IV

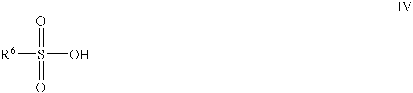

where R⁷ is an organic radical selected from the group comprising unsubstituted and mono- or polysubstituted, branched and straight-chain alkyl radicals, cycloalkyl radicals, alkenyl radicals having one or more double bonds, alkynyl radicals having one or more triple bonds, aryl radicals, alkylaryl radicals, arylalkyl radicals, arylalkenyl radicals, alkyloxyalkyl radicals, hydroxyalkyl radicals and alkylthioalkyl radicals.

In a preferred process, R⁷ is defined as follows: unsubstituted and mono- or polysubstituted, branched and straight-chain (C₁-C₁₈)-alkyl radicals, (C₃-C₁₈)-cycloalkyl radicals, (C₂-C₂₆)-alkenyl radicals having one or more double bonds, (C₂-C₂₆)-alkynyl radicals having one or more triple bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, (C₆-C₁₀)-aryl-(C₂-C₂₆)-alkenyl radicals, (C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals, any substituents being selected from the group comprising OH, OR⁴, NH₂, NHR⁴, NR⁴R⁵, Cl, Br, I and F, where R⁴ and R⁵ are each independently selected from the group comprising H, unsubstituted and mono- or polysubstituted, branched and straight-chain (C₁-C₁₈)-alkyl radicals, (C₃-C₁₈)-cycloalkyl radicals, (C₂-C₂₆)-alkenyl radicals having one or more double bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, especially benzyl radicals,(C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals.

In a preferred process, the organic acid is selected from the group of 1-aminopropylphosphonic acid, aminomethylphosphonic acid, xylenephosphonic acids, phenylphosphonic acid, 1-aminopropylphosphonic acid, toluenephosphonic acid.

In a further process, the organic acid is an alpha-hydroxycarboxylic acid of the general formula Ia

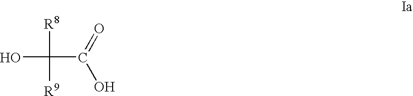

where R⁸ and R⁹ are each independently selected from the group comprising H, OH, OR⁴, NH₂, NHR⁴, NR⁴R⁵, Cl, Br, I, F, unsubstituted and mono- or polysubstituted, branched and straight-chain alkyl radicals, cycloalkyl radicals, alkenyl radicals having one or more double bonds, alkynyl radicals having one or more triple bonds, aryl radicals, alkylaryl radicals, arylalkyl radicals, arylalkenyl radicals, alkyloxyalkyl radicals, hydroxyalkyl radicals and alkylthioalkyl radicals, where R⁴ and R⁵ are each independently selected from the group comprising H, unsubstituted and mono- or polysubstituted, straight-chain and branched (C₁-C₁₈)-alkyl radicals, (C₃-C₁₈)-cycloalkyl radicals, (C₂-C₂₆)-alkenyl radicals having one or more double bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, especially benzyl radicals, (C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals.

It is further preferred that R⁸ and R⁹ are each independently selected from the group of unsubstituted and mono- or polysubstituted, branched and straight-chain (C₁-C₁₈)-alkyl radicals, (C₃-C₁₈)-cycloalkyl radicals, (C₂-C₂₆)-alkenyl radicals having one or more double bonds, (C₂-C₂₆)-alkynyl radicals having one or more triple bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, (C₆-C₁₀)-aryl-(C₂-C₂₆)-alkenyl radicals, (C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals, any substituents being selected from the group comprising OH, OR⁴, NH₂, NHR⁴, NR⁴R⁵, Cl, Br, I and F, where R⁴ and R⁵ are each independently selected from the group comprising H, unsubstituted and mono- or polysubstituted, branched and straight-chain (C₁-C₁₈)-alkyl radicals, (C₃-C₁₈)-cycloalkyl radicals, (C₂-C₂₆)-alkenyl radicals having one or more double bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, especially benzyl radicals,(C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals.

In a preferred process, the organic acid is selected from the group of 2-hydroxyisobutyric acid, 2-hydroxy-4-methylthiobutyric acid, lactic acid, glycolic acid, malic acid, tartaric acid, gluconic acid, glyceric acid.

In a further preferred process, the organic acid is a beta-hydroxycarboxylic acid of the general formula Ib

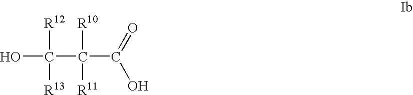

where R¹⁰, R¹¹, R¹² and R¹³ are each independently selected from the group comprising H, OH, OR⁴, NH₂, NHR⁴, NR⁴R⁵, Cl, Br, I, F, unsubstituted and mono- or polysubstituted, branched and straight-chain alkyl radicals, cycloalkyl radicals, alkenyl radicals having one or more double bonds, alkynyl radicals having one or more triple bonds, aryl radicals, alkylaryl radicals, arylalkyl radicals, arylalkenyl radicals, alkyloxyalkyl radicals, hydroxyalkyl radicals and alkylthioalkyl radicals, where R⁴ and R⁵ are each independently selected from the group comprising H, unsubstituted and mono- or polysubstituted, straight-chain and branched (C₁-C₁₈)-alkyl radicals, (C₃-C₁₈)-cycloalkyl radicals, (C₂-C₂₆)-alkenyl radicals having one or more double bonds, (C₆-C₁₀)-aryl radicals, especially phenyl radicals, (C₁-C₁₈)-alkyl-(C₆-C₁₀)-aryl radicals, (C₆-C₁₀)-aryl-(C₁-C₁₈)-alkyl radicals, especially benzyl radicals, (C₁-C₁₈)-alkyloxy-(C₁-C₁₈)-alkyl radicals, (C₁-C₁₈)-hydroxyalkyl radicals and (C₁-C₁₈)-alkylthio-(C₁-C₁₈)-alkyl radicals.

The organic acid is preferably selected from the group of 3-hydroxypropionic acid, 3-hydroxybutyric acid, 3-hydroxyvaleric acid, 3-hydroxyhexanoic acid, 3-hydroxyoctanoic 2 0 acid, 3-hydroxyisobutyric acid. Just like 2-hydroxyisobutyric acid, 3-hydroxyisobutyric acid can likewise serve as a precursor for methacrylic acid and methacrylic esters.

In a further preferred process, the stripping medium or entraining gas used is steam, air, gases, preferably natural gas, methane, oxygen, inert gas, preferably nitrogen, helium, argon, or mixtures thereof.

With regard to the introduction of the stripping medium or entraining gas, an amount of stripping medium or entraining gas, based on the aqueous ammonium salt solution used, between 1 1/kg and 10 000 1/kg, particularly between 10 1/kg and 500 1/kg and very particularly between 20 1/kg and 100 1/kg is preferred.

In further preferred processes, the organic extractant is selected from the group of straight-chain or branched aliphatic ketones having 5 to 18 carbon atoms, heterocyclic ketones having 5 to 18 carbon atoms, straight-chain or branched aliphatic alcohols having 4 to 18 carbon atoms, heterocyclic alcohols having 5 to 18 carbon atoms, straight-chain or branched aliphatic alkanes having 5 to 18 carbon atoms, cycloalkanes having 5 to 14 carbon atoms, straight-chain or branched ethers having 4 to 18 carbon atoms, aromatics substituted by halogen atoms or hydroxyl groups, straight-chain or branched alkanes which are substituted by halogen atoms and have 1 to 18 carbon atoms, cycloalkanes which are substituted by halogen atoms and have 5 to 14 carbon atoms, preferably isobutyl methyl ketone, isopropyl methyl ketone, ethyl methyl ketone, butyl methyl ketone, ethyl propyl ketone, methyl pentyl ketone, ethyl butyl ketone, dipropyl ketone, hexyl methyl ketone, ethyl pentyl ketone, heptyl methyl ketone, dibutyl ketone, 2-undecanone, 2-dodecanone, cyclohexanone, cyclopentanone, 1-butanol, 2-butanol, 1-pentanol, 1-hexanol, 2-hexanol, 3-hexanol, 1-heptanol, 2-heptanol, 3-heptanol, 1-octanol, 2-octanol, 3-octanol, 4-octanol, 1-nonanol, 2-nonanol, 3-nonanol, 5-nonanol, 1-decanol, 2-decanol, 1-undecanol, 2-undecanol, 1-dodecanol, 2-dodecanol, cyclopentanol, cyclohexanol, kerosene, petroleum benzine, pentane, hexane, heptane, octane, nonane, decane, undecane, dodecane, cyclopentane, cyclohexane, cycloheptane, methyl tert-butyl ether, petroleum ether, dibutyl ether, diisopropyl ether, dipropyl ether, diethyl ether, ethyl tert-butyl ether, dipentyl ether, benzene, toluene, o-xylene, m-xylene, p-xylene, chlorobenzene, dichloromethane, chloroform, tetrachloromethane or mixtures thereof.

In further preferred processes, the free acid is obtained from the extractant laden with the extracted acid by a separation process selected from distillation, rectification, crystallization, re-extraction, chromatography, adsorption or membrane processes.

One advantage of the process according to the invention is that of being less expensive, since the expensive workup and/or disposal of the amounts of salt obtained in equimolar amounts is eliminated, and another is that the back-integration of the ammonia released into a production process and the closed circuit of the extractant causes it to work in an environmentally friendly and resource-protective manner.

The use of otherwise much-used assistants, for example sulphuric acid to release the free acid from the ammonium salt, is eliminated, just like additional reaction steps associated with high costs, for example the transamination of the ammonium salt with a secondary or tertiary amine or ester formation with an alcohol and subsequent hydrolysis to the free acid.

The process works in a more energy-saving manner, since the reactive extraction can be performed at lower temperatures than the thermal salt dissociation. Employment of high pressures is usually unnecessary; this lowers the capital costs of an industrial plant. By virtue of the use of a stripping medium or entraining gas, the release of the acid and the extraction thereof succeed within significantly shorter reaction times and with significantly higher yields. The reactive extraction described here is thus more economically viable than the processes described in the prior art.

The novel process described here for releasing acids from the ammonium salts thereof is more economically viable and more environmentally friendly.

### BRIEF DESCRIPTION OF THE DRAWINGS

FIG. 1 shows the schematic setup of the perforator used for reactive extraction.

FIG. 2 shows the schematic setup of the extraction apparatus used (countercurrent extractor).

FIG. 3 shows the schematic setup of a cascade reactive extraction.

FIG. 4 shows the schematic setup of an industrial reactive extraction with high-boiling extractants.

FIG. 5 shows the schematic setup of an industrial reactive extraction with low-boiling extractants.

FIG. 6 shows the influence of the stripping medium on the yield of the free organic acid.

FIG. 7 shows the influence of the temperature on the yield of the free organic acid.

FIG. 8 shows the influence of the starting concentration of the ammonium salt of the organic acid on the yield of the free organic acid in question.

FIG. 9 shows the influence of different extractants on the yield of the free organic acid.

FIG. 10 shows the course of formation of the free acid using the example of lactic acid.

FIG. 11 shows the course of formation of the free acid using the example of 2-hydroxyisobutyric acid.

FIG. 12 shows the course of formation of the free acid using the example of valeric acid.

FIG. 13 shows the course of formation of the free acid using the example of 2-hydroxy-4-methylthiobutyric acid in the countercurrent reactor.

FIG. 14 shows the course of formation of the free acid using the example of 2-hydroxy-4-methylthiobutyric acid in the countercurrent reactor with different amounts of stripping medium introduced.

### DETAILED DESCRIPTION OF THE INVENTION

The invention described here comprises an improved process for releasing a substituted or unsubstituted organic acid, preferably a carboxylic acid (I-III), sulphonic acid (IV) or phosphonic acid (V), more preferably an alpha-hydroxycarboxylic acid (Ia), from the ammonium salt thereof by releasing and removing ammonia and simultaneously extracting the acid released from the aqueous phase with a suitable extractant.

This process corresponds to a reactive extraction. The reactive extraction of an organic acid from the aqueous ammonium salt solution thereof can be improved significantly by the use of a stripping medium or entraining gas, for example nitrogen, air, steam or inert gases, for example argon. The ammonia released is removed from the aqueous solution by the continuous gas stream and can be fed back into a production process. The free acid can be obtained from the extractant by a process such as distillation, rectification, crystallization, re-extraction, chromatography, adsorption, or by a membrane process.

Extraction is understood to mean a separation process in which the enrichment or recovery of substances from mixtures is achieved with the aid of selective solvents or extractants. As in all thermal separation processes, the separation in the extraction is based on the different distribution of mixture components between two or more coexisting phases, which normally arise through the limited miscibility of the individual components with one another (miscibility gap). The mass transfer over the phase interface proceeds through diffusion until a stable end state—the thermodynamic equilibrium—has been established. After equilibrium has been attained, the phases must be separable mechanically. Since these again consist of a plurality of components, further separation processes (for example distillation, crystallization or extraction) for workup are generally connected downstream. In the reactive extraction, at least one reaction is superimposed on the extraction. This influences the thermodynamic equilibria and thus improves the mass transfer between the phases.

It has now been found that the reactive extraction of organic acids such as carboxylic acids, sulphonic acids and phosphonic acids and especially of alpha-hydroxycarboxylic acids from the aqueous ammonium salt solutions thereof can be improved by the use of a stripping medium or entraining gas, for example nitrogen, air, steam or inert gases, for example argon. The ammonia released is removed from the aqueous solution by the continuous gas stream. This shifts the equilibrium of the reaction significantly to the right (Scheme 3, using the example of carboxylic acids).

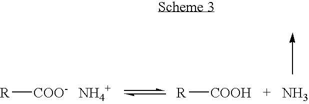

The free organic acid formed is extracted immediately from the aqueous solution by a suitable extractant. This does not cause any significant lowering of the pH of the aqueous solution. This does not hinder the release of further ammonia. The remaining proportion of ammonium salt in the aqueous phase is less than 1%. The organic acid released is extracted completely.

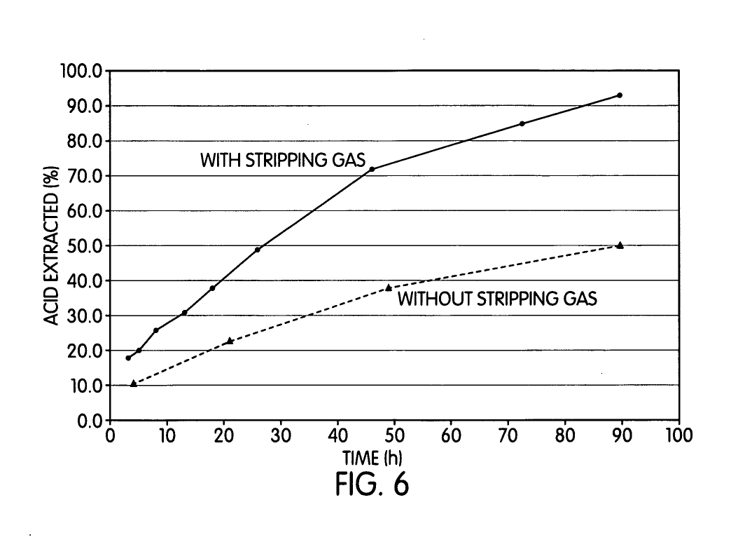

In the case of use of a 10% aqueous 2-hydroxy-4-methylthiobutyric acid ammonium salt solution at 80° C., with isobutyl methyl ketone as the extractant and without entraining gas, after 90 hours of extraction time, 50% of the 2-hydroxy-4-methylthiobutyric acid used was found (Example 5). Under identical conditions, 61 of nitrogen per hour were additionally passed, as an entraining gas for the ammonia released, through the aqueous 2-hydroxy-4-methylthiobutyric acid ammonium salt solution. After 90 hours of extraction time, the proportion of 2-hydroxy-4-methylthiobutyric acid extracted rises to 93% (Example 1, FIG. 6).

It has been found that the temperature has a great influence on the extraction rate. The higher the temperature of the aqueous ammonium salt solution, the more rapidly the reactive extraction proceeds.

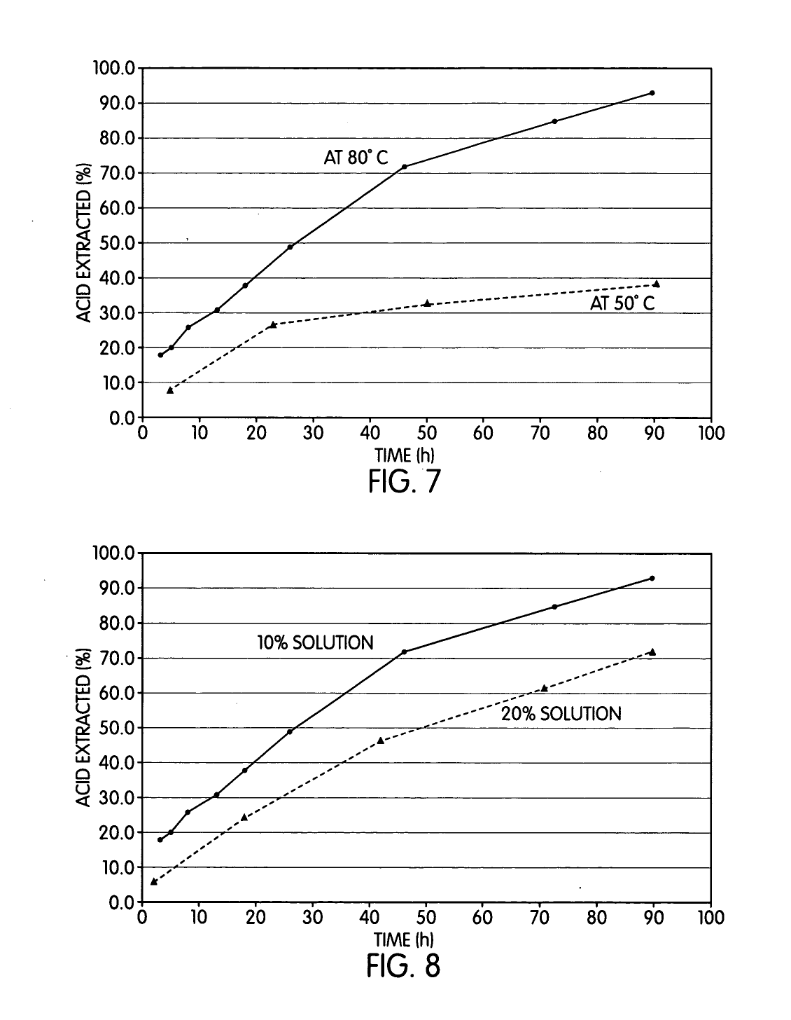

In the case of use of a 10% aqueous 2-hydroxy-4-methylthiobutyric acid ammonium salt solution at 50° C. with isobutyl methyl ketone as the extractant and with 6 1 of nitrogen per hour as the entraining gas, after 90 hours of extraction time, 39% of the 2-hydroxy-4-methylthiobutyric acid used was found (Example 2). A temperature increase by 30° C. to 80° C. under otherwise identical conditions also increases the amount of 2-hydroxy-4-methylthiobutyric acid extracted within the same period to 93% (Example 1, FIG. 7).

It has also been found that the concentration of the ammonium salt used has an influence on the extraction rate. The higher the concentration of the ammonium salt in the aqueous solution, the more slowly the reactive extraction proceeds.

In the case of use of a 10% aqueous 2-hydroxy-4-methylthiobutyric acid ammonium salt solution at 80° C. with isobutyl methyl ketone as the extractant and with 6 1 of nitrogen per hour as the entraining gas, after 90 hours of extraction time, 93% of the 2-hydroxy-4-methylthiobutyric acid used was found (Example 1). When the concentration of the ammonium salt is increased to 20%, under otherwise identical conditions, 71% of extracted 2-hydroxy-4-methylthiobutyric acid is obtained within the same period (Example 3, FIG. 8).

The reactive extraction is not restricted to the use of isobutyl methyl ketone as the extractant. It is possible to use all water-immiscible or only sparingly water-miscible organic solvents, such as alcohols, ethers, ketones or hydrocarbons, or mixtures thereof.

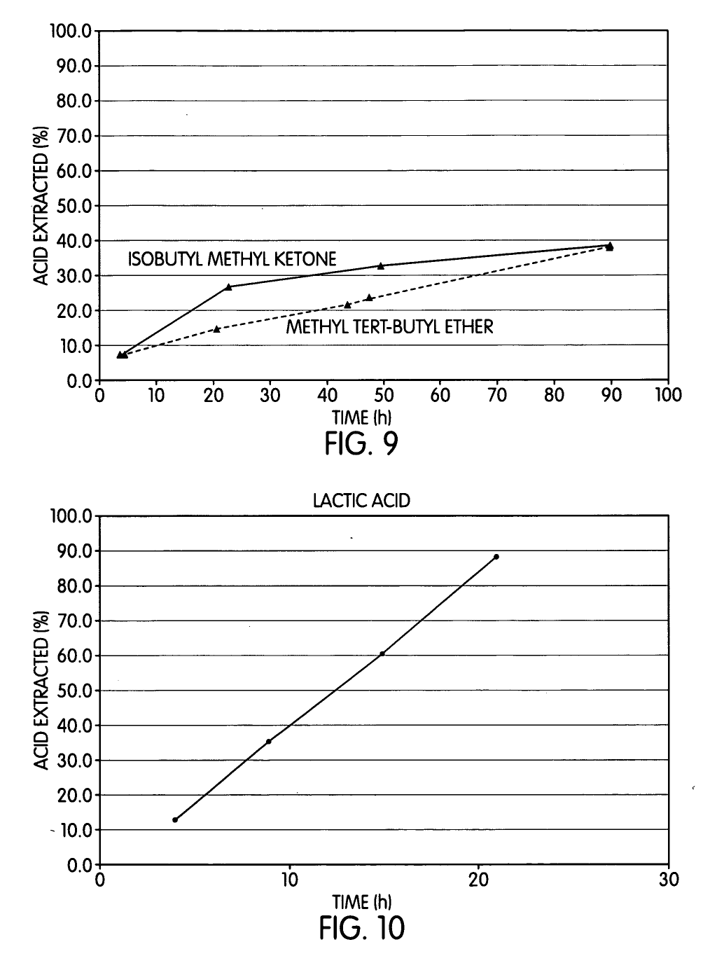

In the case of use of a 10% aqueous 2-hydroxy-4-methylthiobutyric acid ammonium salt solution at 50° C. with isobutyl methyl ketone as the extractant and with 6 1 of nitrogen per hour as the entraining gas, after 90 hours of extraction time, 39% of the 2-hydroxy-4-methylthiobutyric acid used was found (Example 2). When, under identical conditions, methyl tent-butyl ether is used as the extractant, after 90 hours of extraction time, 38% of the 2-hydroxy-4-methylthiobutyric acid used is found in the solvent (Example 4, FIG. 9).

In addition to 2-hydroxy-4-methylthiobutyric acid, reactive extraction using a stripping medium or entraining gas is also employable for other hydroxycarboxylic acids.

Examples cited here include the commercially significant lactic acid, and 2-hydroxyisobutyric acid, which is used in plastics production as a precursor for MMA.

In the case of use of a 10% aqueous lactic acid ammonium salt solution at 80° C. with 1-butanol as the extractant and with 61 of nitrogen per hour as the entraining gas, after 21 hours of extraction time, 88% of the lactic acid used was found (Example 8, FIG. 10).

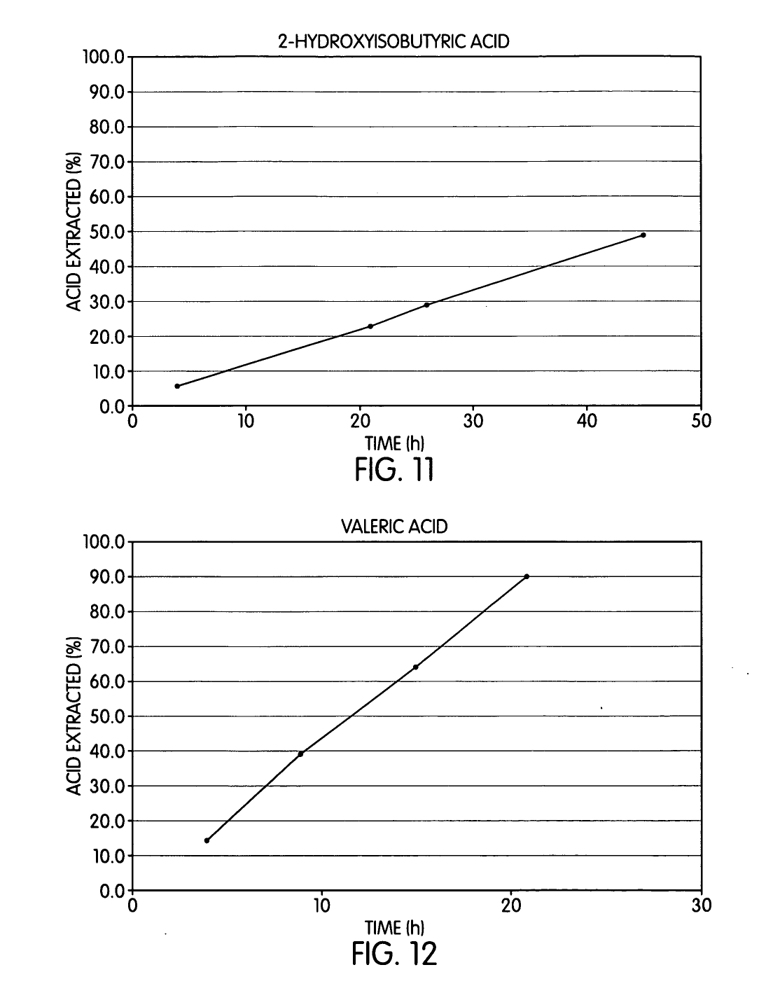

In the case of use of a 10% aqueous 2-hydroxyisobutyric acid ammonium salt solution at 80° C. with isobutyl methyl ketone as the extractant and with 6 1 of nitrogen per hour as the entraining gas, after 21 hours of extraction time, 49% of the 2-hydroxyisobutyric acid used was found (Example 7, FIG. 11).

The invention cited is not restricted only to the release of hydroxycarboxylic acids from the ammonium salts thereof, but also includes other substituted or unsubsubstituted carboxylic acids, e.g. valeric acid, and sulphonic acids, e.g. (+)-camphor-10-sulphonic acid, and phosphonic acids, for example toluenephosphonic acid.

In the case of use of a 10% aqueous valeric acid ammonium salt solution at 80° C. with isobutyl methyl ketone as the extractant and with 6 1 of nitrogen per hour as the entraining gas, after 21 hours of extraction time, 90% of the valeric acid used was found (Example 9, FIG. 12).

In the case of use of a 10% aqueous (+)-camphor-10-sulphonic acid ammonium salt solution at 80° C. with isobutyl methyl ketone as the extractant and with 6 1 of nitrogen per hour as the entraining gas, after 66 hours of extraction time, 25% of the (+)-camphor-10-sulphonic acid used was found (Example 12).

In the case of use of a 10% aqueous toluenephosphonic acid ammonium salt solution, it was possible at 80° C. with isobutyl methyl ketone as the extractant and with 61 of nitrogen per hour as the entraining gas, after 46 hours of extraction time, to find 43% of the toluenephosphonic acid used (Example 13).

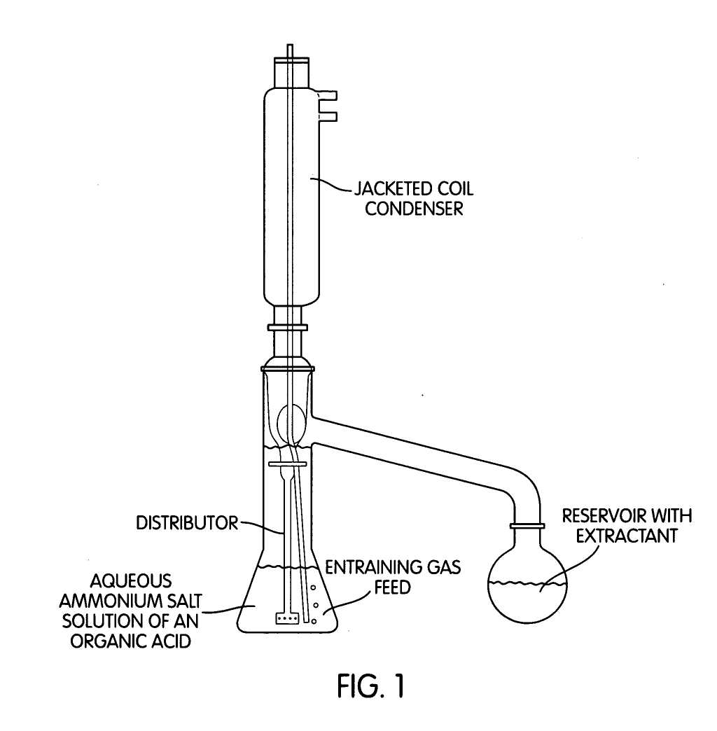

The examples cited were performed in a specially developed perforator (FIG. 1). The extraction vessel of the perforator was half-filled with an aqueous ammonium salt solution of an organic acid and filled with an extractant until there is overflow to the reservoir. The reservoir itself is likewise half-filled with extractant. The extraction vessel is equipped with an inserted distributor and a gas inlet tube with frit. The distributor is rotated by means of magnetic coupling. A stripping gas, for example nitrogen, is simultaneously introduced via the gas inlet tube. The extractant supplied to the distributor from the condenser above through a tube by distillation out of the reservoir is accelerated into the aqueous ammonium salt solution to be extracted as fine droplets out of small holes of a distributor ring by centrifugal force. This achieves fine distribution and intimate mixing of the extractant with the material for extraction. At the same time, the gas stream drives the ammonia out of the aqueous phase. Caused by the co-rotation of the aqueous ammonium salt solution to be extracted, the finely distributed extractant laden with the free organic acid extracted reaches the deposition zone only after prolonged residence time in the aqueous phase and runs back into the reservoir (distillation flask), from which the solvent is recycled into the extraction circuit by re-evaporation. The free organic acid collects in the reservoir. The ammonia stream released is removed with the stripping gas via the attached jacketed coil condenser and collected in an aqueous sulphuric acid trap.

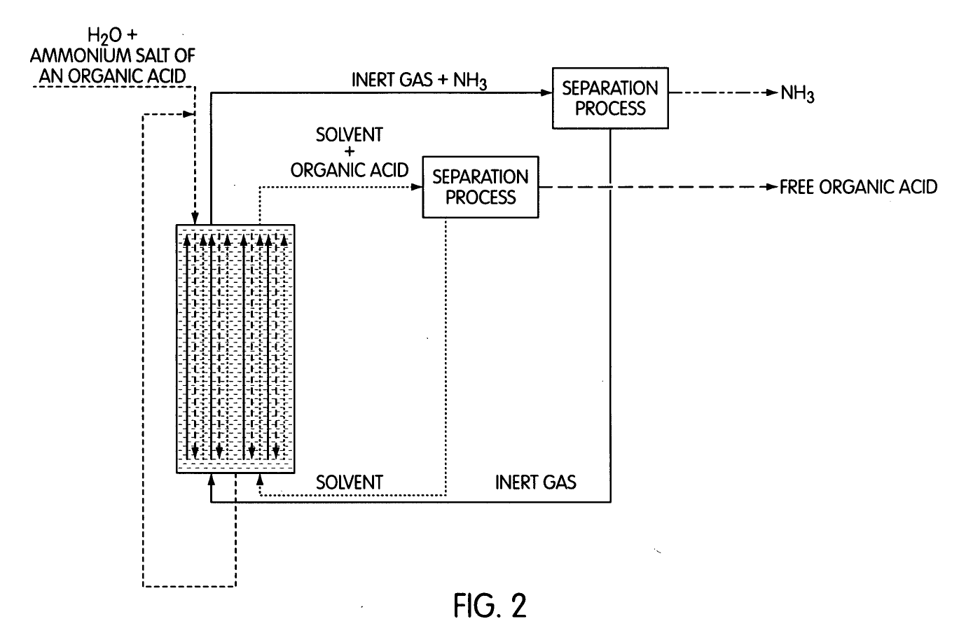

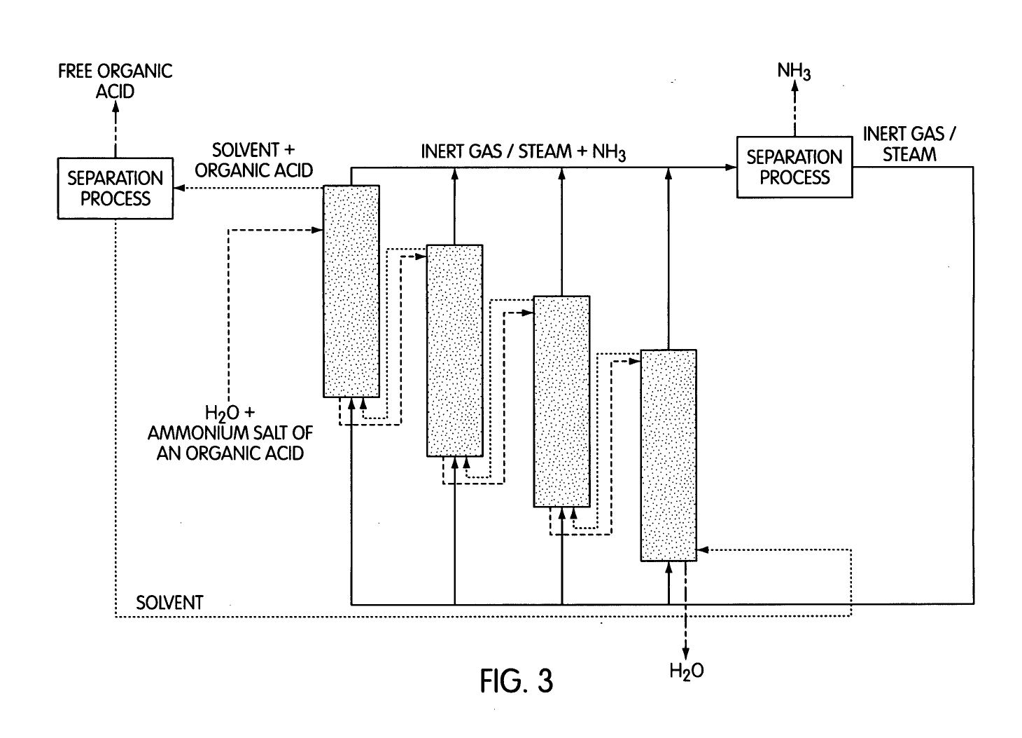

An apparatus improvement is the countercurrent extractor (FIG. 2). In a reaction tube equipped with random packings, the aqueous ammonium salt solution of an organic acid at a controlled temperature is introduced from the top and pumped in circulation. The extractant is pumped into the reaction tube in countercurrent through a frit and the entraining gas is introduced into the system. The finely distributed droplets of the extractant absorb the organic acid released. The lighter organic phase is removed via an outlet at the upper end of the reaction tube. After the separation of extractant and organic acid (for example crystallization, distillation, separation by cooling, separation by back-washing with water), the extractant is introduced into the circuit again. The entraining gas and the released ammonia which has been driven out are removed via the top. Series connection of a plurality of countercurrent extractors makes the process even more efficient and industrially employable. A cascade reactive extraction is obtained (FIG. 3).

The advantage of these two apparatuses (FIGS. 2 and 3) over the perforator is that it is firstly possible here to use significantly higher entraining gas flows and there is secondly continuous removal of the organic acid released. In this way, the extractant can always be reused unburdened and thus dissolve more organic acid released. Redissolution of the organic acid by the water in the ammonium salt solution is thus prevented. The extraction can thus be conducted with high extraction rates.

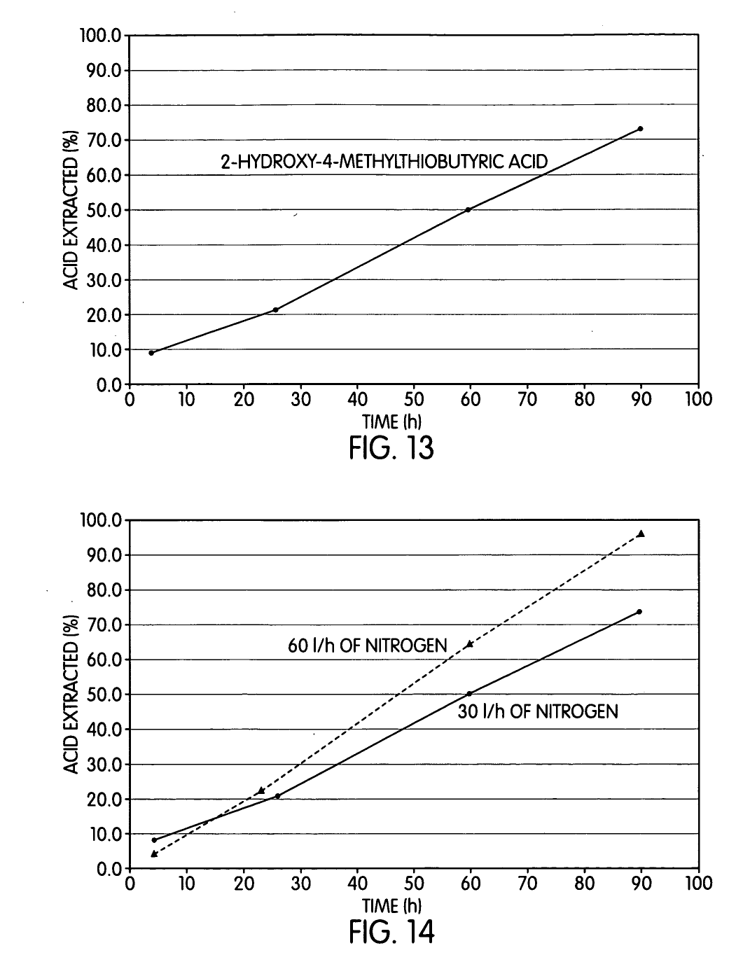

One example cited here is a reactive extraction with 2-hydroxy-4-methylthiobutyric acid and isobutyl methyl ketone. In the case of use of a 10% aqueous 2-hydroxy-4-methylthiobutyric acid ammonium salt solution at 80° C. with isobutyl methyl ketone as the extractant and with 30 1 of nitrogen per hour as the entraining gas, after 90 hours of extraction time, 73% of the 2-hydroxy-4-methylthiobutyric acid used was found (Example 6, FIG. 13).

High influence is possessed here not only by the temperature in the extraction column and the amount of entraining gas introduced but also by the flow rates of the extractant and of the aqueous ammonium salt solution. The temperature is guided by the extractant used and should be below the boiling point of a possible azeotrope.

In the case of use of a 10% aqueous 2-hydroxy-4-methylthiobutyric acid ammonium salt solution at 80° C. with isobutyl methyl ketone as the extractant and with 60 1 of nitrogen per hour as the stripping medium, after 90 hours of extraction time, 95% of the 2-hydroxy-4-methylthiobutyric acid used was found (Example 10, FIG. 14).

Means of Industrial Implementation

An apparatus used on the industrial scale in a liquid-liquid extraction by the countercurrent principle is a mixer-settler apparatus. In countercurrent extraction, in a mixer-settler, the carrier and the extractant are conducted through the mixing battery in opposite directions. Thus, in the first stage, the highly laden carrier stream is contacted with already enriched extractant, which results in a first refining step. With each stage, the loading of the carrier stream decreases. The loading of the extractant stream contacted therewith decreases in the same direction, such that, ultimately, the already highly depleted raffinate is dispersed with fresh unladen extractant in the last stage. In the countercurrent process, a high depletion of the raffinate is achieved with small amounts of extractant, which makes this variant very economically viable.

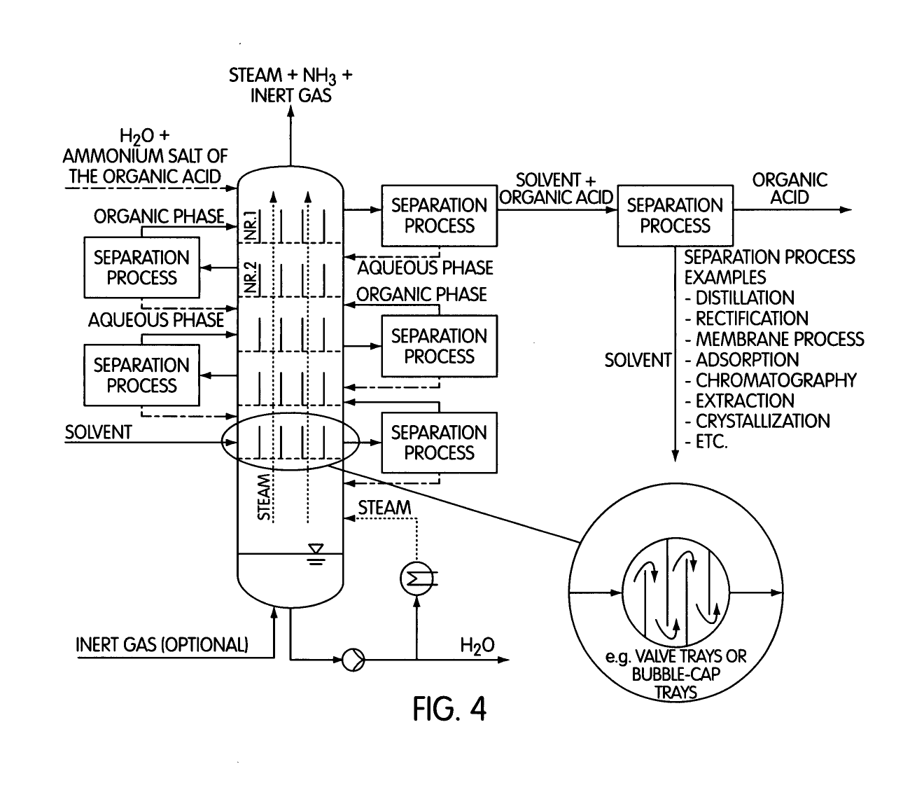

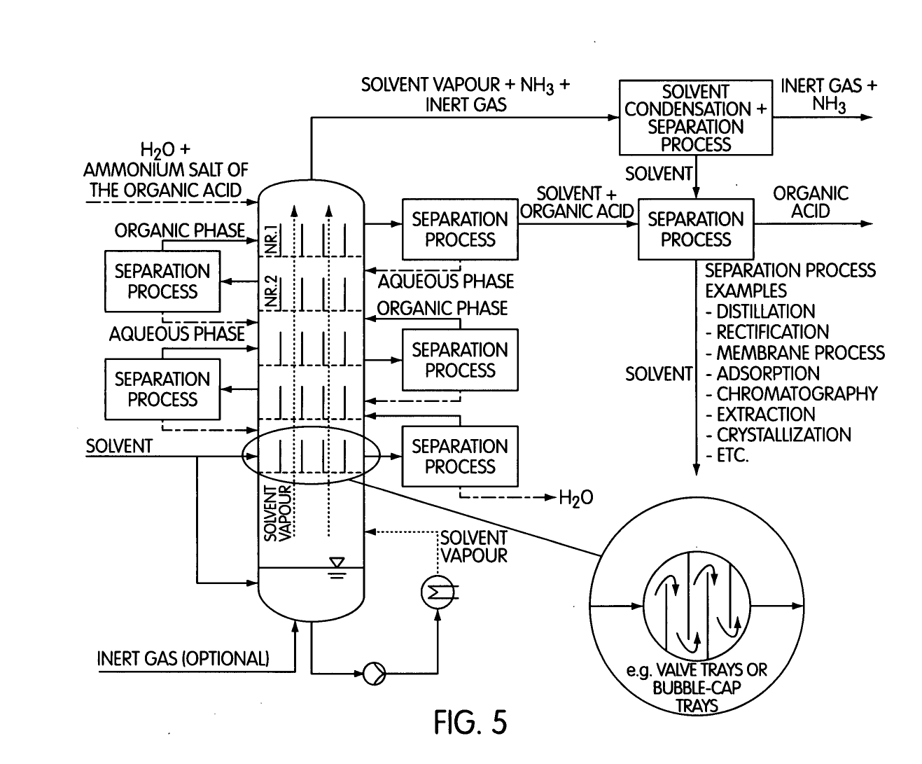

The apparatuses shown (FIG. 4 for high boilers as the extractant and FIG. 5 for low boilers) serve to dissociate ammonium salts of organic acids into ammonia and the corresponding organic acids, it being possible for this thermal dissociation to take place under mild conditions, such that there is no decomposition of the organic acids.

The apparatus consists of a column with n trays, the trays of which are preferably configured as bubble-cap or valve trays, such that there is only a very minor degree of, if any, direct trickle of liquid phases from the upper trays through to the trays below. A stripping medium flows through the column from the bottom upwards, which is preferably introduced into the bottom of the column or below the lowermost tray. The stripping medium may preferably be steam, which is obtained by the heating of the aqueous phase passed downwards, or the stripping medium or entraining gas may also consist of an inert gas, for example nitrogen or another gas which forms a mixture with ammonia by interactions, which is readily convertible to the gas phase.

The configuration of the trays is preferably such that the aqueous and organic phases are conducted together from the entry to the tray to the exit by suitable baffles, in order to prevent back-mixing or short-circuit flows. On the trays, by virtue of the gas entering at the bottom, good mixing of all three phases takes place, such that the ammonia released by thermal decomposition, owing to the large phase interface, is converted easily to the gas phase and the organic acid which forms can be extracted rapidly out of the aqueous phase into the organic phase.

Thus, the aqueous phase comprising the ammonium salt and the organic phase which absorbs the organic acid are firstly introduced together to the uppermost tray (No. 1) of the column and mixed. In the case of an extraction in countercurrent, the aqueous phase with the greatest ammonium salt loading is combined on the uppermost tray with the organic phase which absorbs the organic acid downstream of the separation process belonging to the tray below (No. 2). As a result of the thermal elimination of ammonia, by virtue of the contact with the gas phase, it passes into the gas phase at each tray, and the organic acid passes from the aqueous phase into the organic phase. If ammonium salt is present in the aqueous phase, no thermal equilibrium will exist between the concentration of the organic acid in the aqueous phase and in the organic phase, and as a result there is always transfer of the organic acid which forms after the elimination of ammonia into the organic phase. After the aqueous and organic phases have left the uppermost tray, the two phases are separated from one another in a suitable separation process. In the case of low mutual solubility of organic and aqueous phases, this separation process may be a phase separator. In order to promote the phase separation, a temperature change can be effected before the separation process. Further separation processes, such as a distillation, rectification, membrane process, crystallization, adsorption, chromatography, etc., are likewise possible. Thus, at the uppermost tray, the organic phase with the highest loading of the organic acid is obtained. The organic acid can be separated from the solvent by one or more further separation processes such as a distillation, rectification, membrane process, crystallization, adsorption, chromatography, etc. The solvent released can then be fed back into the column for extraction.

After the separation process on the uppermost tray (No. 1), the aqueous phase passes to the tray below (No. 2) and is in turn combined with the organic phase which absorbs the organic acid downstream of the separation process belonging to the tray below (No. 3). The fresh organic solvent, or that recycled from preceding separation processes, is combined on the lowermost tray (No. N) together with the aqueous phase from the tray above.

To operate such a reactive extraction, it is necessary to decide whether water or the organic solvent possesses a higher boiling temperature. In the case of water as the lower-boiling component at the operating pressure of the column, and of utilization of steam as the entraining medium, the water is collected in the lower part of the column and evaporated by means of a heat exchanger operated in circulation. This evaporator may also be configured as a natural circulation evaporator. The excess water is conducted out of the column under fill level control. The steam is introduced again below the lowermost tray (No. N). Steam, ammonia and optionally a further gas and small amounts of solvent vapours are passed out at the top of the column and can optionally be separated in a downstream separation process.

In the case of water as the higher-boiling component at the operating pressure of the column, the organic solvent can also be evaporated according to the above description and be used as an entraining medium. To this end, the solvent is either added fresh under fill level control to the lower part of the column, or recycled from the separation processes for solvent removal from the organic acid or solvent removal from the top stream. Solvent vapours, ammonia and possibly a further gas and small amounts of steam are passed out at the top of the column and can optionally be separated in a downstream separation process. In this case, water is discharged from the process after the separation process on the lowermost tray (No. N). In the case of relatively large solvent losses to the gas phase, it is possible to add fresh or recycled solvent at each tray, in order to balance out this loss.

All of said processes of the present invention are preferably performed in an aqueous medium. In addition, the processes of the present invention can be performed in batch processes known to those skilled in the art or in continuous processes.

Separation Processes

In order to separate the free organic acid from the extractant on completion of extraction, various processes are employable:

For example, the extractant laden with the free acid can be cooled in a phase separator. The free organic acid separates out as a more highly concentrated aqueous phase with the water dissolved in the extractant and can be removed thus. After distillative removal of the water, the free acid is present in pure form. The extractant can be fed directly back into the extraction circuit.

Distillative removal of the extractant is also possible. The extractant laden with the free acid is heated to boiling and distilled off at standard pressure or reduced pressure in a distillation apparatus of customary design. This distillate, which contains water in the case of an azeotrope-forming solvent or else is anhydrous, can be fed directly back into the extraction circuit. The free acid remains in the distillation bottoms.

A further means of removing the free organic acid from the laden extractant is re-extraction with water. To this end, the extractant laden with the free organic acid is re-extracted from the organic solvent with water in a countercurrent extraction in an extraction apparatus (e.g. FIG. 2). According to the degree of extraction, a one-stage or multistage extraction is necessary. The organic extractant which is now unladen again can be fed directly back into the extraction circuit. The aqueous solution of the free organic acid can be concentrated to the desired concentration by distillative removal of the water. The above mentioned separation processes were tested successfully with 2-hydroxy-4-methylthiobutyric acid as the model compound.

According to the type of organic acid used, the removal from the organic extractant can also be effected by crystallization, adsorption, membrane processes, chromatography, rectification, or the like.

### Examples

### Example 1

Extraction of MHA from a 10% MHA ammonium salt solution with isobutyl methyl ketone in a rotational perforator at 80° C. (inventive) 17.6 g (90 mmol, M=167.2 g/mol, with a content of 85.1%) of MHA ammonium salt were dissolved in 132.4 g of water. This 10% salt solution was initially charged in the rotational perforator (FIG. 1) and heated to 80° C. The solvent flask was initially charged with 500 g of isobutyl methyl ketone which were heated to boiling (internal temperature 115-117° C.). 6 1 of nitrogen per hour were passed continuously through the aqueous salt solution. During the reaction time, analysis samples were taken from the solvent flask and analysed for dissolved MHA by HPLC. After 90 hours, the extraction was ended and the yellow-coloured isobutyl methyl ketone and the aqueous phase were weighed and analysed by HPLC. 6% of the MHA used was still found in the aqueous phase, 93% in the isobutyl methyl ketone. An ion chromatography analysis of the organic phase showed an ammonium content of <100 ppm.

### Example 2

Extraction of MHA from a 10% MHA ammonium salt solution with isobutyl methyl ketone in a rotational perforator at 50° C. (inventive) 16.3 g (90 mmol, M=167.2 g/mol, with a content of 92.3%) of MHA ammonium salt were dissolved in 133.7 g of water. This 10% salt solution was initially charged in the rotational perforator (FIG. 1) and heated to 50° C. The solvent flask was initially charged with 500 g of isobutyl methyl ketone which were heated to boiling (internal temperature 115-117° C.). 6 1 of nitrogen per hour were passed continuously through the aqueous salt solution. During the reaction time, analysis samples were taken from the solvent flask and analysed for dissolved MHA by HPLC. After 90 hours, the extraction was ended and the yellow-coloured isobutyl methyl ketone and the aqueous phase were weighed and analysed by HPLC. 60% of the MHA used was still found in the aqueous phase, 39% in the isobutyl methyl ketone. An ion chromatography analysis of the organic phase showed an ammonium content of <100 ppm.

### Example 3

Extraction of MHA from a 20% MHA ammonium salt solution with isobutyl methyl ketone in a rotational perforator (inventive) 32.6 g (180 mmol, M=167.2 g/mol, with a content of 92.3%) of MHA ammonium salt were dissolved in 117.4 g of water. This 20% salt solution was initially charged in the rotational perforator (FIG. 1) and heated to 80° C. The solvent flask was initially charged with 500 g of isobutyl methyl ketone which were heated to boiling (internal temperature 115-117° C.). 6 1 of nitrogen per hour were passed continuously through the aqueous salt solution. During the reaction time, analysis samples were taken from the solvent flask and analysed for dissolved MHA by HPLC. After 90 hours, the extraction was ended and the yellow-coloured isobutyl methyl ketone and the aqueous phase were weighed and analysed by HPLC. 28% of the MHA used was still found in the aqueous phase, 71% in the isobutyl methyl ketone. An ion chromatography analysis of the organic phase showed an ammonium content of <100 ppm.

### Example 4

Extraction of MHA from a 10% MHA ammonium salt solution with methyl tert-butyl ether (MTBE) in a rotational perforator (inventive) 16.3 g (90 mmol, M=167.2 g/mol, with a content of 92.3%) of MHA ammonium salt were dissolved in 133.7 g of water. This 10% salt solution was initially charged in the rotational perforator (FIG. 1) and heated to 50° C. The solvent flask was initially charged with 500 g of methyl tert-butyl ether which were heated to boiling (internal temperature 55-56° C.). 6 1 of nitrogen per hour were passed continuously through the aqueous salt solution. During the reaction time, analysis samples were taken from the solvent flask and analysed for dissolved MHA by HPLC. After 90 hours, the extraction was ended and the yellow-coloured methyl tert-butyl ether and the aqueous phase were weighed and analysed by HPLC. 71% of the MHA used was still found in the aqueous phase, 38% in the methyl tert-butyl ether. An ion chromatography analysis of the organic phase showed an ammonium content of <100 ppm.

### Example 5

Extraction of MHA from a 10% MHA ammonium salt solution with isobutyl methyl ketone in a rotational perforator at 80° C. (noninventive) 16.3 g (90 mmol, M=167.2 g/mol, with a content of 92.3%) of MHA ammonium salt were dissolved in 133.7 g of water. This 10% salt solution was initially charged in the rotational perforator (FIG. 1) and heated to 80° C. The solvent flask was initially charged with 500 g of isobutyl methyl ketone which were heated to boiling (internal temperature 115-117° C.). During the reaction time, analysis samples were taken from the solvent flask and analysed for dissolved MHA by HPLC. After 90 hours, the extraction was ended and the yellow-coloured isobutyl methyl ketone and the aqueous phase were weighed and analysed by HPLC. 49% of the MHA used was still found in the aqueous phase, 50% in the isobutyl methyl ketone. An ion chromatography analysis of the organic phase showed an ammonium content of <100 ppm.

### Example 6

Extraction of MHA from a 10% MHA ammonium salt solution with isobutyl methyl ketone in a countercurrent extractor (inventive) 43.3 g (239 mmol, M=167.2 g/mol, with a content of 92.3%) of MHA ammonium salt were dissolved in 356.7 g of water. This 10% salt solution was initially charged in the reservoir vessel of the countercurrent extractor (FIG. 2) and heated to 80° C. 1333 g of isobutyl methyl ketone were likewise heated to 80° C. in the solvent reservoir. The extractor column was filled at the start with aqueous salt solution. During the extraction, 30 1 of nitrogen per hour were allowed to bubble continuously through the liquid column. Both liquid circulations were kept constant over the total extraction time. The aqueous salt solution was pumped in circulation at 5 ml/min and the isobutyl methyl ketone at 8 ml/min. The effluxing MHA-containing isobutyl methyl ketone phase was passed into the distillation vessel via the heated phase separator (80° C.). The isobutyl methyl ketone which was distilled off under gentle conditions was fed back into the circuit via the solvent reservoir. The MHA extracted remained in the distillation flask with isobutyl methyl ketone which had not been distilled off. After 90 hours, the extraction was ended and the yellow-coloured isobutyl methyl ketone from the distillation and the aqueous phase were weighed and analysed by HPLC. 26% of the MHA used was still found in the aqueous phase, 73% in the isobutyl methyl ketone. An ion chromatography analysis of the organic phase showed an ammonium content of <100 ppm.

### Example 7

Extraction of 2-hydroxyisobutyric acid from a 10% 2-hydroxyisobutyric acid ammonium salt solution in a specific rotational perforator using an entraining gas as the stripping medium (inventive) 13.0 g (124 mmol, M=104.1 g/mol, with a content of 99%) of 2-hydroxyisobutyric acid were initially charged in 130.4 g of water and admixed with 6.6 g of 32% aqueous ammonia solution (0.124 mol). This 10% salt solution was initially charged in the rotational perforator (FIG. 1) and heated to 80° C. The solvent flask was initially charged with 500 g of isobutyl methyl ketone which were heated to boiling (internal temperature 115-117° C.). 6 1 of nitrogen per hour were passed continuously through the aqueous salt solution. During the reaction time, analysis samples were taken from the solvent flask and analysed for dissolved 2-hydroxyisobutyric acid by HPLC. After 45 hours, the extraction was ended and the pale-coloured isobutyl methyl ketone and the aqueous phase were weighed and analysed by HPLC. 50% of the 2-hydroxyisobutyric acid used was still found in the aqueous phase, 49% in the isobutyl methyl ketone. An ion chromatography analysis of the organic phase showed an ammonium content of <100 ppm.

### Example 8

Extraction of lactic acid from a 10% lactic acid ammonium salt solution in a specific rotational perforator using an entraining gas as the stripping medium (inventive) 8.1 g (90 mmol, M=90.08 g/mol, with a content of 99%) of lactic acid were initially charged in 50 g of water and admixed with 6.5 ml of 32% aqueous ammonia solution (0.1 mol). This 10% salt solution was initially charged in the rotational perforator (FIG. 1) and heated to 80° C. The solvent flask was initially charged with 500 g of 1-butanol saturated with water at boiling temperature, which were heated to boiling (internal temperature 97-99° C.). 6 1 of nitrogen per hour were passed continuously through the aqueous salt solution. During the reaction time, analysis samples were taken from the solvent flask and analysed for dissolved lactic acid by HPLC. After 21 hours, the extraction was ended and the pale-coloured 1-butanol and the aqueous phase were weighed and analysed by HPLC. 11% of the lactic acid used was still found in the aqueous phase, 88% in the 1-butanol. An ion chromatography analysis of the organic phase showed an ammonium content of <100 ppm.

### Example 9

Extraction of valeric acid from a 10% valeric acid ammonium salt solution in a rotational perforator (inventive) 9.3 g (90 mmol, M=102.13 g/mol, with a content of 99%) of valeric acid were initially charged in 50 g of water and admixed with 6.5 ml 32% aqueous ammonia solution (0.1 mol). After stirring for 30 minutes, the excess ammonia and most of the water were drawn off from the clear colourless solution at 40° C. in a water-jet vacuum. The resulting oil (16.7 g) was dissolved in 98.4 g of water. This 10% salt solution was initially charged in the rotational perforator (FIG. 1) and heated to 80° C. The solvent flask was initially charged with 500 g of isobutyl methyl ketone which were heated to boiling (internal temperature 115-117° C.). 6 1 of nitrogen per hour were passed continuously through the aqueous salt solution. During the reaction time, analysis samples were taken from the solvent flask and analysed for dissolved valeric acid by GC. After 21 hours, the extraction was ended and the pale-coloured isobutyl methyl ketone and the aqueous phase were weighed and analysed by HPLC. 9% of the valeric acid used was still found in the aqueous phase, 90% in the isobutyl methyl ketone. An ion chromatography analysis of the organic phase showed an ammonium content of <100 ppm.

### Example 10

Extraction of MHA from a 10% MHA ammonium salt solution with isobutyl methyl ketone in a countercurrent extractor (inventive) 43.3 g (239 mmol, M=167.2 g/mol, with a content of 92.3%) of MHA ammonium salt were dissolved in 356.7 g of water. This 10% salt solution was initially charged in the reservoir vessel of the countercurrent extractor (FIG. 2) and heated to 80° C. 1333 g of isobutyl methyl ketone were likewise heated to 80° C. in the solvent reservoir. The extractor column was filled at the start with aqueous salt solution. During the extraction, 60 1 of nitrogen per hour were allowed to bubble continuously through the liquid column. Both liquid circulations were kept constant over the total extraction time. The aqueous salt solution was pumped in circulation at 5 ml/min and the isobutyl methyl ketone at 8 ml/min. The effluxing MHA-containing isobutyl methyl ketone phase was passed into the distillation vessel via the heated phase separator (80° C.). The isobutyl methyl ketone which was distilled off under gentle conditions was fed back into the circuit via the solvent reservoir. The MHA extracted remained in the distillation flask with isobutyl methyl ketone which had not been distilled off. After 90 hours, the extraction was ended and the yellow-coloured isobutyl methyl ketone from the distillation and the aqueous phase were weighed and analysed by HPLC. 4% of the MHA used was still found in the aqueous phase, 95% in the isobutyl methyl ketone. An ion chromatography analysis of the organic phase showed an ammonium content of <100 ppm.

### Example 12

Extraction of (+)-camphor-10-sulphonic acid from a 10% (+)-camphor-10-sulphonic acid ammonium salt solution with isobutyl methyl ketone in a specific rotational perforator using an entraining gas as the stripping medium (inventive) 21.3 g (90 mmol, M=232.30 g/mol, with a content of 98%) of (+)-camphor-10-sulphonic acid were initially charged in 50 g of water and admixed with 6.5 ml of 32% aqueous ammonia solution (0.1 mol). After stirring for 30 minutes, the excess ammonia and most of the water were drawn off from the clear colourless solution at 40° C. in a water-jet vacuum. The resulting white solid (39.8 g) was dissolved in 209.5 g of water. This 10% salt solution was initially charged in the rotational perforator (FIG. 1) and heated to 80° C. The solvent flask was initially charged with 500 g of isobutyl methyl ketone which were heated to boiling (internal temperature 115-117° C.). 6 1 of nitrogen per hour were passed continuously through the aqueous salt solution. After 66 hours, the extraction was ended and the pale-coloured isobutyl methyl ketone and the aqueous phase were concentrated to dryness. 74% of the (+)-camphor-10-sulphonic acid used was still found in the aqueous phase, 25% in the isobutyl methyl ketone. An ion chromatography analysis of the organic phase showed an ammonium content of <100 ppm.

### Example 13

Extraction of toluenephosphonic acid from a 10% toluenephosphonic acid ammonium salt solution with isobutyl methyl ketone in a specific rotational perforator using an entraining gas as the stripping medium (inventive) 20.0 g (113.9 mmol, M=172.12 g/mol, with a content of 98%) of toluenephosphonic acid were initially charged in 50 g of water and admixed with 8.5 ml of 32% aqueous ammonia solution (0.13 mol). After stirring for 30 minutes, the excess ammonia and most of the water were drawn off from the clear colourless solution at 40° C. in a water jet vacuum. The resulting oil (24.5 g) was dissolved in 190.9 g of water. This 10% salt solution was initially charged in the rotational perforator (FIG. 1) and heated to 80° C. The solvent flask was initially charged with 500 g of isobutyl methyl ketone which were heated to boiling (internal temperature 115-117° C.). 6 1 of nitrogen per hour were passed continuously through the aqueous salt solution. After 23 hours, the extraction was ended and the pale-coloured isobutyl methyl ketone and the aqueous phase were concentrated to dryness. 56% of the toluenephosphonic acid used was still found in the aqueous phase, 43% in the isobutyl methyl ketone. An ion chromatography analysis of the organic phase showed an ammonium content of <100 ppm.

1-19. (canceled)

20. A process for converting an ammonium salt of an organic acid to a free organic acid, comprising:
a) contacting an aqueous solution of the ammonium salt with an organic extractant;
b) dissociating said ammonium salt at a temperature and pressure at which the aqueous solution and the extractant are in a liquid state; and
c) introducing a stripping medium or entraining gas in order to remove NH₃ from the aqueous solution and transfer at least a portion of the free organic acid formed to the organic extractant.

21. The process of claim 20, wherein the conversion is carried out at a pressure of 0.01 bar to 20 bar.

22. The process of claim 21, wherein the salt dissociation is performed at a temperature of 20° C. to 300° C.

23. The process of claim 20, wherein:
a) the conversion is carried out at a pressure of 0.01 bar to 200 bar;
b) salt dissociation is performed at a temperature of 5° C. to 300° C.; and
c) the starting concentration of the ammonium salt of an organic acid in the aqueous solution used is in the range of from 90% by weight to 1% by weight.

24. The process of claim 23, wherein the extractant used is a sparingly water-miscible or entirely water-immiscible solvent.

25. The process of claim 24, wherein the weight ratio of aqueous solution to organic extractant is from 1:100 to 100:1.

26. The process 20, wherein the organic acid is selected from the group consisting of: monocarboxylic acid; dicarboxylic acid; tricarboxylic acid; ascorbic acid;
sulphonic acid; phosphonic acid; and hydroxycarboxylic acid.

27. The process of claim 26, wherein,
a) the conversion is carried out at a pressure of 0.1 bar to 5 bar;
b) salt dissociation is performed at a temperature of 50° C. to 130° C.;
c) the starting concentration of the ammonium salt of an organic acid in the aqueous solution used is in the range of from 60% by weight to 10% by weight;
d) the weight ratio of aqueous solution to organic extractant is from 1:5 to 5:1; and
e) after the salt dissociation has ended, the organic acid formed is obtained from the organic extractant.

28. The process of claim 20, wherein the organic acid is a carboxylic acid of general formula I
X¹—COOH I
where X¹ is an organic radical selected from the group consisting of: unsubstituted and mono- or polysubstituted, branched and straight-chain alkyl radicals; cycloalkyl radicals; alkenyl radicals having one or more double bonds; alkynyl radicals having one or more triple bonds; aryl radicals; alkylaryl radicals; arylalkyl radicals; arylalkenyl radicals; alkyloxyalkyl radicals; hydroxyalkyl radicals; and alkylthioalkyl radicals.

29. The process of claim 20, wherein said organic acid is selected from the group consisting of: acetic acid; propionic acid; butyric acid; valeric acid; caproic acid; oenanthic acid; caprylic acid; pelargonic acid; capric acid; lauric acid; palmitic acid; stearic acid; omega-3 fatty acids; omega-6 fatty acids; omega-9 fatty acids; salicylic acid; benzoic acid; ferulic acid; cinnamic acid; vanillic acid; gallic acid; hydroxycinnamic acid; hydroxybenzoic acid; 3-hydroxypropionic acid;
succinic acid; oxalic acid; malonic acid; glutaric acid; adipic acid; pimelic acid; suberic acid; azelaic acid; sebacic acid; fumaric acid; itaconic acid; methylmalonic acid; phthalic acid; terephthalic acid; isophthalic acid;
citric acid; cyclopentane-1,2,3-tricarboxylic acid; cyclopentane-1,2,4-tricarboxylic acid; 2-methylcyclopentane-1,2,3-tricarboxylic acid; 3-methylcyclopentane-1,2,4-tricarboxylic acid;
p-toluenesulphonic acid; camphor-10-sulphonic acid; benzenesulphonic acid; dodecylbenzenesulphonic acid; naphthalenesulphonic acids; phenolsulphonic acids;
1-aminopropylphosphonic acid; aminomethylphosphonic acid; xylenephosphonic acids; phenylphosphonic acid; 1-aminopropylphosphonic acid; toluenephosphonic acid;
2-hydroxyisobutyric acid; 2-hydroxy-4-methylthiobutyric acid; lactic acid; glycolic acid; malic acid; tartaric acid; gluconic acid; glyceric acid;
3-hydroxypropionic acid; 3-hydroxybutyric acid; 3-hydroxyvaleric acid; 3-hydroxyhexanoic acid; 3-hydroxyoctanoic acid; and 3-hydroxyisobutyric acid.

30. The process of claim 20, wherein the organic acid is a dicarboxylic acid of general formula II
HOOC—X²—COOH II
where X² is an organic radical selected from the group consisting of: unsubstituted and mono- or polysubstituted, branched and straight-chain alkanediyl radicals; cycloalkanediyl radicals; alkenediyl radicals having one or more double bonds; alkynediyl radicals having one or more triple bonds; aryldiyl radicals; alkylaryldiyl radicals; arylalkanediyl radicals; arylalkenediyl radicals; alkyloxyalkanediyl radicals; hydroxyalkanediyl radicals; and alkylthioalkanediyl radicals.

31. The process of claim 20, wherein the organic acid is a tricarboxylic acid of general formula III 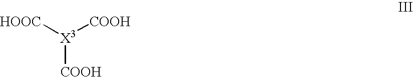
where X³ is an organic radical selected from the group consisting of: unsubstituted and mono- or polysubstituted, branched and straight-chain alkanetriyl radicals; cycloalkanetriyl radicals; alkenetriyl radicals having one or more double bonds; alkynetriyl radicals having one or more triple bonds; aryltriyl radicals; alkylaryltriyl radicals; arylalkanetriyl radicals; arylalkenetriyl radicals; alkyloxyalkanetriyl radicals; hydroxyalkanetriyl radicals; and alkylthioalkanetriyl radicals.

32. The process of claim 20, wherein the organic acid is a sulphonic acid of general formula IV 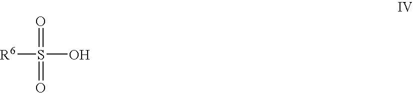
where R⁶ is an organic radical selected from the group consisting of: unsubstituted and mono- or polysubstituted, branched and straight-chain alkyl radicals; cycloalkyl radicals; alkenyl radicals having one or more double bonds; alkynyl radicals having one or more triple bonds; aryl radicals; alkylaryl radicals; arylalkyl radicals;
arylalkenyl radicals; alkyloxyalkyl radicals; hydroxyalkyl radicals; and alkylthioalkyl radicals.

33. The process of claim 20, wherein the organic acid is a phosphonic acid of general formula V 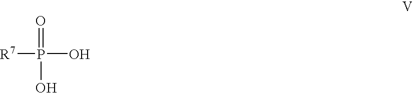
where R⁷ is an organic radical selected from the group consisting of: unsubstituted and mono- or polysubstituted, branched and straight-chain alkyl radicals; cycloalkyl radicals; alkenyl radicals having one or more double bonds; alkynyl radicals having one or more triple bonds; aryl radicals; alkylaryl radicals; arylalkyl radicals; arylalkenyl radicals; alkyloxyalkyl radicals; hydroxyalkyl radicals; and alkylthioalkyl radicals.

34. The process of claim 20, wherein the organic acid is an alpha-hydroxycarboxylic acid of general formula Ia 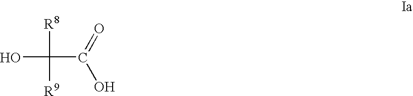
where R⁸ and R⁹ are each independently selected from the group consisting of: H; OH; OR⁴; NH₂; NHR⁴; NR⁴R⁵; Cl; Br; I; F; unsubstituted and mono- or polysubstituted, branched and straight-chain alkyl radicals; cycloalkyl radicals;
alkenyl radicals having one or more double bonds; alkynyl radicals having one or more triple bonds; aryl radicals; alkylaryl radicals; arylalkyl radicals; arylalkenyl radicals; alkyloxyalkyl radicals; hydroxyalkyl radicals; and alkylthioalkyl radicals;
where R⁴ and R⁵ are each independently selected from the group comprising H; unsubstituted and mono- or polysubstituted, straight-chain and branched (C₁-C₁₈)-alkyl radicals; (C₃-C₁₈)-cycloalkyl radicals; (C₂-C₂₆)-alkenyl radicals having one or more double bonds; (C₆-C₁₀)-aryl radicals; (C₁-C₁₈)-alkyl-(C₆-C₁₀)-aryl radicals; (C₆-C₁₀)-aryl-(C₁-C₁₈)-alkyl radicals; (C₁-C₁₈)-alkyloxy-(C₁-C₁₈)-alkyl hydroxyalkyl radicals; and (C₁-C₁₈)-alkylthio-(C₁-C₁₈)-alkyl radicals.

35. The process of claim 20, wherein the organic acid is a beta-hydroxycarboxylic acid of general formula Ib 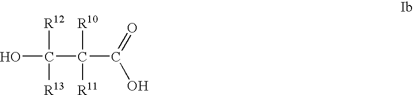
where R¹⁰, R¹¹, R¹² and R¹³ are each independently selected from the group consisting of: H; OH; OR⁴; NH₂; NHR⁴; NR⁴R⁵; Cl; Br; I; F; unsubstituted and mono- or polysubstituted, branched and straight-chain alkyl radicals; cycloalkyl radicals; alkenyl radicals having one or more double bonds; alkynyl radicals having one or more triple bonds; aryl radicals; alkylaryl radicals; arylalkyl radicals; arylalkenyl radicals; alkyloxyalkyl radicals; hydroxyalkyl radicals; and alkylthioalkyl radicals; where R⁴ and R⁵ are each independently selected from the group consisting of: H; unsubstituted and mono- or polysubstituted, straight-chain and branched (C₁-C₁₈)-alkyl radicals; (C₃-C₁₈)-cycloalkyl radicals; (C₂-C₂₆)-alkenyl radicals having one or more double bonds; (C₆-C₁₀)-aryl radicals; (C₁-C₁₈)-alkyl-(C₆-C₁₀)-aryl radicals; (C₆-C₁₀)-aryl-(C₁-C₁₈)-alkyl radicals; (C₁-C₁₈)-alkyloxy-(C₁-C₁₈)-alkyl radicals; (C₁-C₁₈)-hydroxyalkyl radicals; and (C₁-C₁₈)-alkylthio-(C₁-C₁₈)-alkyl radicals.

36. The process of claim 29, wherein the stripping medium or entraining gas used is steam, air, natural gas, methane, oxygen, inert gas, or mixtures thereof

37. The process of claim 36, wherein the amount of stripping medium or entraining gas in relation to the aqueous ammonium salt solution is between 10 1/kg and 500 1/kg.

38. The process of claim 37, wherein the extractant used is selected from the group consisting of: straight-chain or branched aliphatic ketones having 5 to 18 carbon atoms; heterocyclic ketones having 5 to 18 carbon atoms; straight-chain or branched aliphatic alcohols having 4 to 18 carbon atoms; heterocyclic alcohols having 5 to 18 carbon atoms; straight-chain or branched aliphatic alkanes having 5 to 18 carbon atoms; cycloalkanes having 5 to 14 carbon atoms; straight-chain or branched ethers having 4 to 18 carbon atoms; aromatics substituted by halogen atoms or hydroxyl groups; straight-chain or branched alkanes which are substituted by halogen atoms and have 1 to 18 carbon atoms; and cycloalkanes which are substituted by halogen atoms and have 5 to 14 carbon atoms.

39. The process of claim 29, wherein
a) the free acid is obtained from the extractant laden with the extracted acid by a separation process selected from the group consisting of: distillation, rectification; crystallization; re-extraction; chromatography; adsorption; membrane processes; and
b) the extractant used is selected from the group consisting of: isobutyl methyl ketone; isopropyl methyl ketone; ethyl methyl ketone; butyl methyl ketone; ethyl propyl ketone; methyl pentyl ketone; ethyl butyl ketone; dipropyl ketone; hexyl methyl ketone; ethyl pentyl ketone; heptyl methyl ketone; dibutyl ketone; 2-undecanone; 2-dodecanone; cyclohexanone; cyclopentanone; 1-butanol; 2-butanol; 1-pentanol; 1-hexanol; 2-hexanol; 3-hexanol; 1-heptanol; 2-heptanol; 3-heptanol; 1-octanol; 2-octanol; 3-octanol; 4-octanol; 1-nonanol; 2-nonanol; 3-nonanol; 5-nonanol; 1-decanol; 2-decanol; 1-undecanol; 2-undecanol; 1-dodecanol; 2-dodecanol; cyclopentanol; cyclohexanol; kerosene; petroleum benzine; pentane; hexane; heptane; octane; nonane; decane; undecane; dodecane; cyclopentane; cyclohexane; cycloheptane; methyl tert-butyl ether; petroleum ether; dibutyl ether; diisopropyl ether; dipropyl ether; diethyl ether; ethyl tert-butyl ether; dipentyl ether; benzene; toluene; o-xylene; m-xylene; p-xylene; chlorobenzene; dichloromethane; chloroform; tetrachloromethane; and mixtures thereof.
